(19) United States
(12) Patent Application Publication
(10) Pub. No.: US 2025/0072895 A1
(43) Pub. Date: Mar. 6, 2025

(54) SYSTEMS, DEVICES, AND RELATED METHODS FOR FASTENING TISSUE

(71) Applicants: Boston Scientific Scimed, Inc., Maple Grove, MN (US); Multi-Scale Medical Robotics Center Limited, Pak Shek Kok (HK)

(72) Inventors: Barry WEITZNER, Acton, MA (US); Philip Wai Yan CHIU, Shatin (HK); Kai Fung CHAN, Shatin (HK); Haojin YANG, Shatin (HK); Yanfei CAO, Shatin (HK); Kin Wai CHAN, Shatin (HK)

(73) Assignees: Boston Scientific Scimed, Inc., Maple Grove, MN (US); Multi-Scale Medical Robotics Center Limited, Pak Shek Kok (HK)

(21) Appl. No.: 18/818,127

(22) Filed: Aug. 28, 2024

Related U.S. Application Data
(60) Provisional application No. 63/535,738, filed on Aug. 31, 2023.

(51) Int. Cl. A61B 17/072; A61B 17/00; A61B 34/00

(52) U.S. Cl. CPC A61B 17/07207; A61B 34/71; A61B 2017/00398; A61B 2017/00438; A61B 2017/07257; A61B 2017/07271; A61B 2017/07278; A61B 2017/07285; A61B 2034/715

(57) ABSTRACT

In examples, a medical device for tissue fastening may comprise an end effector having: a cartridge, a sled positioned in the cartridge, a first control member coupled to the sled, and a second control member coupled to the sled; a shaft coupled to the end effector; and an operation portion coupled to the shaft. The operation portion may include an actuator that is configured to actuate at least the first control member or the second control member in order to move the sled proximally or distally.

### CROSS-REFERENCE TO RELATED APPLICATIONS

This application claims the benefit of priority under 35 U.S.C. § 119 from U.S. Provisional Application No. 63/535,738, filed Aug. 31, 2023, which is incorporated by reference herein in its entirety.

### TECHNICAL FIELD

This disclosure relates generally to tissue closure. More particularly, at least some embodiments of this disclosure relate to a stapling device or system, for example an endoscopic stapler, and related methods of using the stapling mechanism.

### BACKGROUND

Tissue fastening (e.g., stapling) is used in many medical procedures. These procedures often involve resecting portions or sections of tissue, followed by closing using staples. An example of a common procedure is colorectal anastomosis. In hybrid surgeries where physicians use laparoscopic and endoscopic platforms to conduct a procedure, a rigid stapler is often used. Linear, rigid staplers include long rigid members, which are incapable of being navigated through tortuous anatomy without causing trauma to the tissue. Therefore, there is a need for endoscopic staplers and associated methods.

### SUMMARY

Aspects of this disclosure relate to, among other things, systems, devices, and methods for fastening tissue. Each of the aspects disclosed herein may include one or more of the features described in connection with any of the other disclosed aspects.

In examples, a medical device for tissue fastening may comprise an end effector having: a cartridge, a sled positioned in the cartridge, a first control member coupled to the sled, and a second control member coupled to the sled; a shaft coupled to the end effector; and an operation portion coupled to the shaft. The operation portion may include an actuator that is configured to actuate at least the first control member or the second control member in order to move the sled proximally or distally.

Any of the devices disclosed herein may have any of the following features, alone or in any combination. The actuator may be configured to move the first control member proximally and the second control member distally in order to move the sled proximally. The actuator may be configured to move the first control member distally and the second control member proximally in order to move the sled distally. The first control member and the second control member may be coupled to one another at their distal ends. The first control member may extend along a top surface of a wall of a body of the end effector. The second control member may extend along a bottom surface of the wall of the body. The body may be configured to removably receive the cartridge. The first control member may be fixedly coupled to a bottom surface of the sled. The sled may include a blade and a slider. The slider may be configured to deliver a fastener to a tissue of a subject. The actuator may be a first actuator. The operation portion may include a second actuator. The second actuator may be configured to articulate the shaft. The second actuator may be fixedly coupled to a first sheath of the shaft. At least portions of the first sheath may be movable with respect to a second sheath of the shaft. The operation portion may include a motor. The actuator may be controlled by the motor. The motor may be a first motor. The actuator may be a first actuator. The operation portion includes a second motor that controls a second actuator, wherein the first actuator is configured to actuate the first control member, and wherein the second actuator is configured to actuate the second control member. The operation portion may include a third motor that controls a third actuator. The third actuator is configured to actuate a third control member that is coupled to the end effector to close the end effector. The operation portion may include a fourth motor that is coupled to the shaft and is configured to rotate the shaft. One or more of the first, the second, or the third motors may be configured to accommodate a changing length of at least one of the first control member, the second control member, or the third control member. The operation portion may be releasably coupled to the shaft.

In another example, a medical device for tissue fastening may comprise an end effector having: a cartridge, a sled positioned in the cartridge, and a cable coupled to the sled. The cable may have two proximal ends and a looped distal end. The medical device may further comprise a shaft coupled to the end effector and an operation portion coupled to the shaft. The operation portion may include an actuator that is coupled to the proximal ends of the cable in order to actuate the sled proximally and distally. The cable may be a first cable. The actuator may be fixedly coupled to a second cable. The second cable may have two distal ends and a looped proximal end.

Any of the examples disclosed herein may include any of the following features, alone or in any combination. The distal ends of the second cable may be coupled to the proximal ends of the first cable.

In another example, a medical device for tissue fastening may comprise: an end effector having: an anvil and a cartridge that contains a plurality of fasteners. The medical device may further include a flexible shaft coupled to the end effector and an operation portion releasably coupled to the flexible shaft.

Any of the examples disclosed herein may have any of the following features, alone or in any combination. The operation portion may include at least one motor for controlling the end effector.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIG. 1 depicts an exemplary medical device.

FIG. 2A depicts an end effector of the exemplary medical device.

FIG. 2B shows a first cross-section of the end effector of FIG. 2A.

FIGS. 3A and 3B show a second cross-section of the end effector of FIG. 2A in a first configuration and a second configuration, respectively.

FIG. 4 shows an exemplary sled of the end effector of FIGS. 2A-3B.

FIG. 5 shows a handle and a shaft of the medical device of FIG. 1.

FIGS. 6A-6D show aspects of the handle of FIG. 5.

FIG. 6E shows details of the shaft of FIG. 5.

FIG. 7 shows an alternative medical device.

FIGS. 8A and 8B show aspects of a connection mechanism for use with the medical devices of FIGS. 1-7.

FIG. 9 shows aspects of a system for actuating a medical device, such as the medical devices of FIGS. 1-7.

### DETAILED DESCRIPTION

This disclosure is drawn to systems, devices, and methods for coupling, cutting, and resecting tissue, among other aspects. Reference will now be made in detail to aspects of this disclosure, examples of which are illustrated in the accompanying drawings. Wherever possible, the same or similar reference numbers will be used throughout the drawings to refer to the same or like parts. The term “distal” refers to a portion further away from a user when introducing a device into a subject. By contrast, the term “proximal” refers to a portion closer to the user when placing the device into the subject. The term “tissue fastening” may refer, for example, to stapling, fixing, attaching, fastening, or otherwise joining two portions of tissue together. The term “fastener” may include staples, clips, elastic bands, suture, or any other fastener known in the art. Directions, such as “up,” “down,” “top,” “bottom,” “left,” “right,” and the like refer to an orientation as shown in the figures, even if a device is capable of being reoriented.

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” As used herein, the terms “about,” “substantially,” and “approximately,” indicate a range of values within +/−10% of the stated value unless otherwise stated.

Aspects disclosed herein may include a handle, a flexible shaft, and an end effector (e.g., stapler head). The end effector may include a sled that includes both a slider (e.g., a ramp assembly) for delivering staples and a blade for cutting tissue. The sled may be coupled to two wires or wire segments, such that the sled may be actuated proximally and distally. The handle or other operation portion may include actuators for, among other features, opening/closing the end effector, actuating the sled (both proximally and distally), articulating the shaft and/or rotating the end effector about a longitudinal axis of the device. The shaft may be removably coupled to the handle, such that portion(s) of the medical device (e.g., the handle or a motorized operation portion) may be reused and other portion(s) of the medical device (e.g., the shaft and end effector) may be disposed. In some aspects, the end effector, shaft, and/or other elements of the medical device may be actuated by one or more motors. Such motors may be configured to provide actuation through the flexible shaft of the medical device.

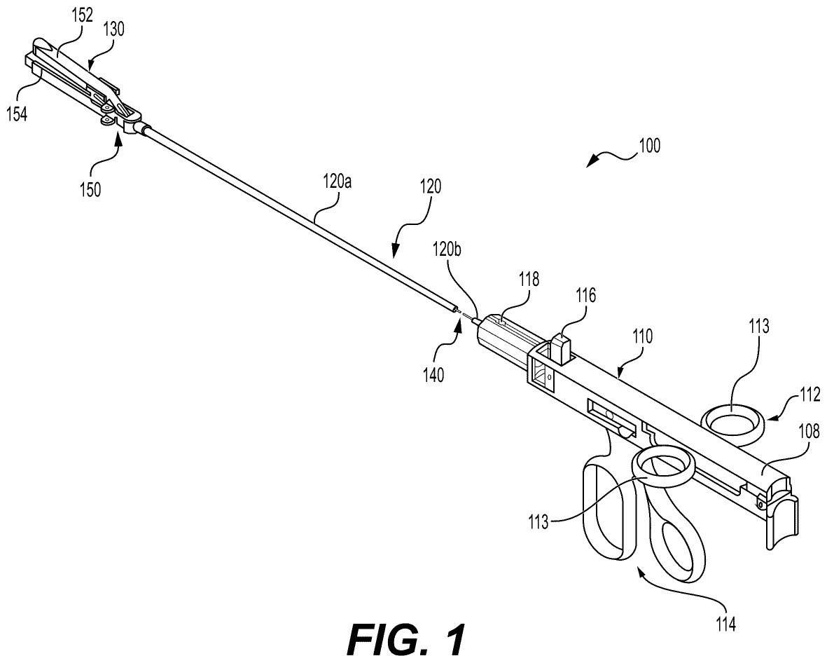

FIGS. 1-6E illustrate aspects of an exemplary medical device 100 that may be used to staple tissue of a subject. In some embodiments, medical device 100 may be a surgical stapling apparatus configured to engage body tissue, apply one or more surgical fasteners (e.g., staplers) thereto, and optionally form an incision in the fastened body tissue during minimally invasive surgical procedures, such as endoscopic procedures. Medical device 100 may be used to apply surgical clips or other fasteners, but will be primarily discussed in the context of applying staples.

As shown particularly in FIG. 1, medical device 100 may include a handle 110 (an operation portion), a shaft 120, and an end effector 130. As described below, handle 110 may include one or more actuators for actuating aspects of shaft 120 and/or end effector 130. As shown in FIG. 1 and discussed in detail below, portions of shaft 120 may be removably coupled to handle 110 via a connection assembly 140. Shaft 120 may include a distal portion 120a, coupled to end effector 130, and a proximal portion 120b, coupled to handle 110. Thus, portions of medical device 100 may be reusable, while other portions of medical device 100 may be disposable. For example, handle 110 may be reusable, while distal portion 120a of shaft 120 and end effector 130 may be disposable (e.g., single-use). Additionally or alternatively, removable coupling of distal portion 120a of shaft 120 to handle 110 may facilitate backfeeding of distal portion 120a into a working channel of an introduction device, such as a scope (e.g., an endoscope). For example, a proximal end of distal portion 120a may be inserted into a distal opening of a working channel of an endoscope, fed proximally through the working channel, and then coupled to handle 120.

Shaft 120 may be any suitable endoscopic member configured to bend and/or articulate so as to traverse tortuous anatomy in a body. Shaft 120 may be formed from one or more biocompatible materials, such as, e.g., HDPE, silicone, polyurethane, ETFE, SIBS, PIB-PUR, or any other suitable medical grade polymers, and may be flexible and configured to extend through tortuous anatomy. Shaft 120 may extend any length suitable for endoscopic or laparoscopic procedures, and may be configured to be positioned within a working channel of an endoscope. Alternatively, shaft 120 may be positioned in the body without an endoscope. Although endoscopes are referenced herein, reference to endoscopes or endoscopy should not be construed as limiting the possible applications of the disclosed aspects. For example, the disclosed aspects may be used with duodenoscopes, bronchoscopes, ureteroscopes, colonoscopes, catheters, diagnostic or therapeutic tools or devices, or other types of medical devices.

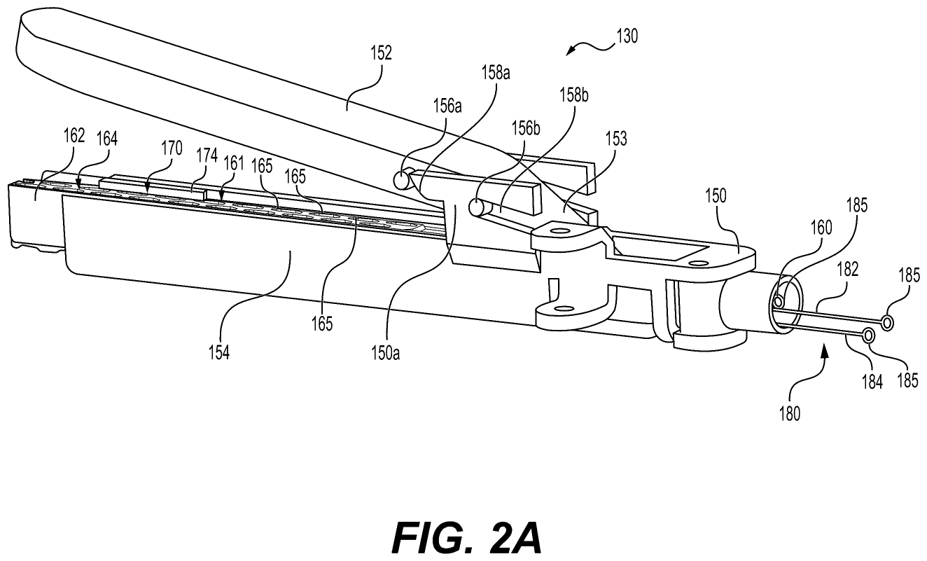

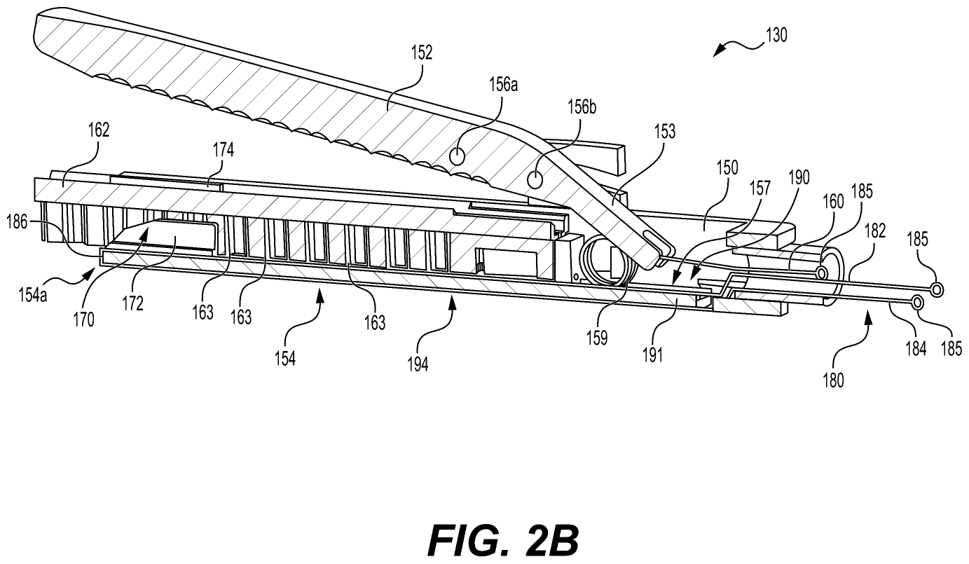

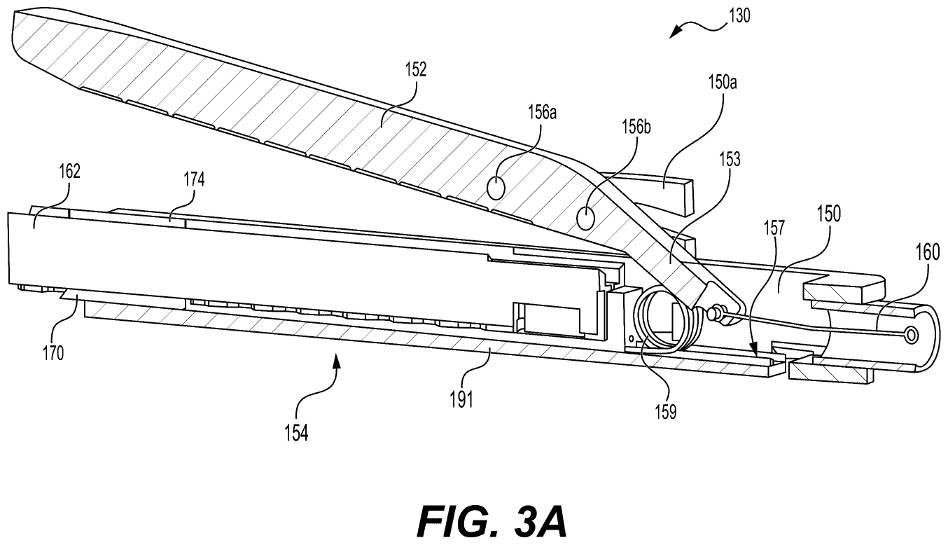

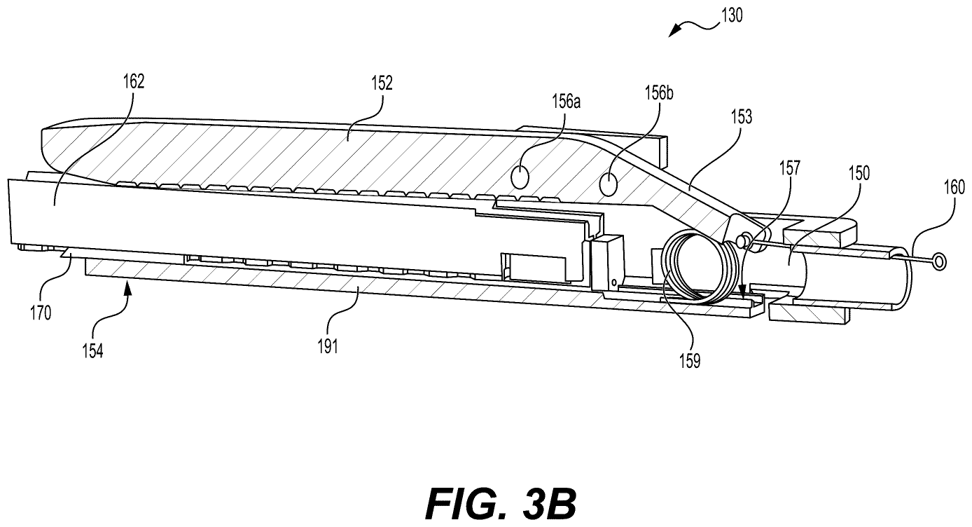

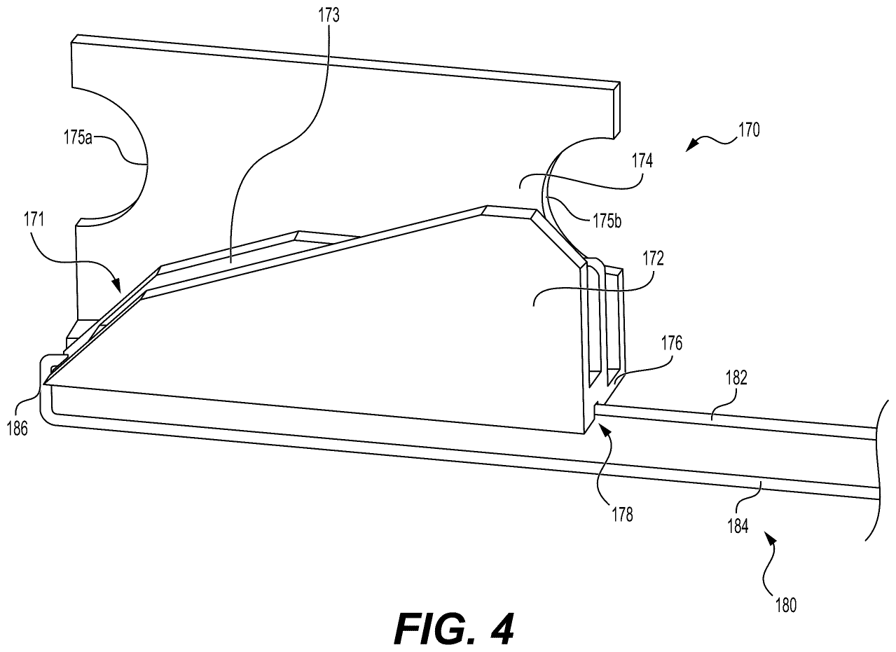

Aspects of end effector 130 are shown in detail in FIGS. 2A-4. End effector 130 is shown and described as a stapler head. However, it will be appreciated that end effector 130 may alternatively be another type of end effector for delivering fasteners to tissue. FIG. 2A shows end effector 130 in an at least partially opened configuration, and FIG. 2B shows a first cross-sectional view of end effector 130 in the same partially opened configuration of FIG. 2A. FIGS. 3A and 3B show other cross-sectional views of end effector 130 in an at least partially open configuration (FIG. 3A) and a closed configuration (FIG. 3B). FIG. 4 shows details of a sled 170 of end effector 130.

End effector 130 may include a base 150 and an anvil 152 (a clip) rotatably and/or pivotably coupled to base 150. Base 150 may include a distally extending portion 154, which may be configured to detachably or fixedly receive a cartridge 162. Anvil 152 may form a first arm, and distally extending portion 154 may form a second arm of end effector 130. In some examples, cartridge 162 may be press-fit into a cavity of base 150. Cartridge 162 may contain and/or have received therein a plurality of staples 163 or other fasteners, as shown in FIG. 2B. As shown in FIG. 2A, a top surface 164 of cartridge 162 (a surface facing anvil 152) may have a plurality of openings 165 formed therein, such that staples 163 may be deployed through openings 165 to secure tissue when tissue is received between cartridge 162 and anvil 152.

A wire or cable 160 (or other type of control member) may be coupled to a proximal end 153 of anvil 152, as shown particularly in FIG. 2B. Although the term “cable” is used herein to describe various structures, it will be appreciated that these structures may be other types of control/actuation mechanisms (e.g., wires, sliders, linkages, bars, etc.). Furthermore, any of the cables disclosed herein may be a segment that may be combined with other control members of the same or different types. When cable 160 is moved proximally or distally, anvil 152 may rotate or pivot relative to base 150. For example, proximal movement of cable 160 may rotate anvil 152 downward, such that end effector 130 transitions to a closed configuration (shown in FIG. 3B). Distal movement of cable 160 may result in or otherwise occur from anvil 152 rotating upward (e.g., via a resilient member 159, discussed below), such that end effector 130 transitions to an open configuration. In some examples, anvil 152 may include one or more pegs 156a, 156b, which may constrain a movement of anvil 152 to a desired rotational path. For example, as shown in FIG. 2A, first, distal peg 156a may interact with a distal surface 158a of an upwardly projecting portion 150a of base 150. A second, proximal peg 156b may be received within and move along a slot 158b of upwardly projecting portion 150a of base 150.

A resilient member 159 may be disposed between base 150 and anvil 152. Resilient member 159 may be, for example, a torsional spring. A first portion of resilient member 159 may be coupled (e.g., fixedly couple via, e.g., bonding) to a bottom surface of anvil 152. A second portion of resilient member 159 may be received within a slot 157 of base 150. Slot 157 may be formed in or on a top surface 190 of a bottom wall 191 of base 150. Resilient member 159 may be movable within but constrained by slot 157, such that anvil 152 is able to transition to a closed configuration (which is shown in FIG. 3B). As shown in FIGS. 3A and 3B, resilient member 159 may be relatively proximal in a closed configuration of end effector 130 (FIG. 3B) as compared to an open configuration of end effector 130 (FIG. 3A). Resilient member 159 may bias end effector 130 to a resting position, such as an open position (FIG. 3A). When cable 160 is moved or otherwise urged proximally, anvil 152 may exert a force on resilient member 159 so that end effector 130 is able to close (FIG. 3B). When cable 160 is released, resilient member 159 may exert a restoring force on anvil 152 such that anvil 152 opens. Cable 160 may automatically move distally when it is released, for example, based on the restoring force exerted by resilient member 159.

Sled 170 may be received within cartridge 162. As shown in FIG. 4, sled 170 may include a slider 171 on one side of sled 170. Slider 171 may include angled actuating portion 172 (e.g., an angled wall). Slider 171 may further include a center wall 173. Center wall 173 may extend parallel to angled actuating portion 172. As sled 170 moves through cartridge 162 (e.g., distally through cartridge 162), angled actuating portion 172 (and/or center wall 173) may engage one or more of staples 163 contained in cartridge 162, pushing staples 163 out of openings 165 and into tissue received between anvil 152 and cartridge 162 in order to deliver staples 163 to tissue of a subject. In some examples, center wall 173 may not actuate staples 163. In other examples, center wall 173 may have any of the features of angled actuating portion 172 and may similarly function to actuate staples.

An opposite side of sled 170 from slider 171 (e.g., on the opposite side of center wall 173) may include cutting member such as a blade 174 (e.g., a knife). A base 176 of sled 170 may extend between slider 171 and blade 174. In some examples, base 176 may be flat and may be approximately perpendicular to angled actuating portion 172, center wall 173, and blade 174. Angled actuating portion 172, center wall 173, and/or blade 174 may be approximately parallel to one another. In some examples, slider 171, blade 174, and base 176 may be formed from a single, unitary piece of material. Alternatively, slider 171 and blade 174 may have alternative arrangements relative to one another. In some examples, before cartridge 162 is inserted into base 150, slider 171 and blade 174 may be inserted into slots on a bottom surface of cartridge 162 (not shown). Gaps/slots between angled actuating portion 172, center wall 173, and/or blade 174 may facilitate the assembly of the sled 170 into cartridge 162.

Blade 174 may have a double-sided design. For example, as shown, blade 174 may include a semicircular (or other shaped) sharpened portions 175a, 175b on both proximal and distal sides of blade 174. Sharpened portions 175a, 175b may be recessed relative to portions of blade 174 that are above and below sharpened portions 175a, 175b. In other words, sharpened portions 175a, 175b may form semicircular (or other shaped) gaps/recesses. Sharpened portions 175a, 175b may enable cutting as blade 174 moves in both proximal and distal directions. In alternatives, sharpened portions 175a, 175b may be omitted, and proximal and distal edges of blade 174 may be sharp so as to cut tissue as blade 175 moves proximally and distally.

As shown in FIG. 2A, for example, blade 174 may extend through a slot 161 on an upper surface of cartridge 162. As sled 170 moves longitudinally/axially through cartridge 162, blade 174 may similarly move longitudinally/axially along slot 161. Movement of blade 174 may cut tissue received between anvil 152 and cartridge 162. Movement of sled 170 may both deploy staples 163 and cut tissue. Thus, fewer actuating cables (discussed below) may be necessary to move sled 170 than if blade 174 and slider 171 were separate elements. Sled 170 may move smoothly along the longitudinal axis of cartridge 162, and friction between cartridge 162 and base 150 may help to prevent the movement (e.g., sliding) of cartridge 162 relative to base 150 during the motion of the sled 170.

Base 176 of sled 170 may include a channel or groove 178 formed therein, as shown in FIG. 4. A first wire or cable 182 may be received within groove 178. For example, first cable 182 may be fixedly attached to groove 178 (e.g., via adhesive, soldering, or other mechanisms). A second wire or cable 184 may be coupled to first cable 182 at a distal connection point 186. Although first cable 182 and second cable 184 are referenced herein, it will be appreciated that first cable 182 and second cable 184 may form a single cable 180, which is folded or loops at connection point 186. Alternatively, first cable 182 and second cable 184 may be separate bodies that are coupled to one another at connection point 186. First cable 182 and second cable 184 are omitted from FIGS. 3A and 3B for ease of showing other features of end effector 130. Cables 182, 184 may be control members.

As shown particularly in FIG. 2B, first cable 182 may extend through/pass through slot 157 of top surface 190. Slot 157 or a slot in communication therewith may extend along distally extending portion 154 of base 150. Second cable 184 may extend at least partially along a bottom surface 194 of bottom wall 191 of base 150 (including along distally extending portion 154). Connection point 186 may wrap around a distal end 154a of distally extending portion 154 (which may be a portion of bottom wall 191). It will be appreciated that, as cables 182, 184 move, relative locations of cables 182, 184 may change. For example, a portion of cable 184 that is along bottom surface 194 of distally extending portion 154 in one configuration (e.g., a configuration in which sled 170 is relatively distal) may be within slot 157 of in another configuration (e.g., a configuration in which sled 170 is relatively proximal). The term first cable 182 may generally apply to the cable that is affixed to sled 170, and the term second cable 184 may generally apply to the cable that is distal to and/or below sled 170. It will be appreciated that connection point 186 (e.g., a looped or folded portion) may not be the actual junction between first cable 182 and second cable 184, depending on the configuration of sled 170. In some examples, first cable 182, second cable 184, and connection point 186 may be a single, unitary structure. In all configurations, an overall shape of such a single cable 180 may be the same or nearly the same, as it wraps around distally extending portion 154, with ends of cable 180 in different positions depending on a configuration of sled 170.

As first cable 182 moves proximally, second cable 184 may move distally, and sled 170 may move proximally. As second cable 184 moves proximally, first cable 182 may move distally, and sled 170 may move distally. Movement of sled 170 may thus be achieved by pulling proximally on one of first cable 182 or second cable 184.

Each of cables 160, 182, 184 may include a respective ring 185 at a proximal, free end thereof. Each of rings 185 may be fixed to another respective cable (e.g., a cable of shaft 120 or a cable of handle 110). Rings 185 may facilitate exchange of end effector 130 with other elements (e.g., other end effectors) to be used with shaft 120 and/or handle 110.

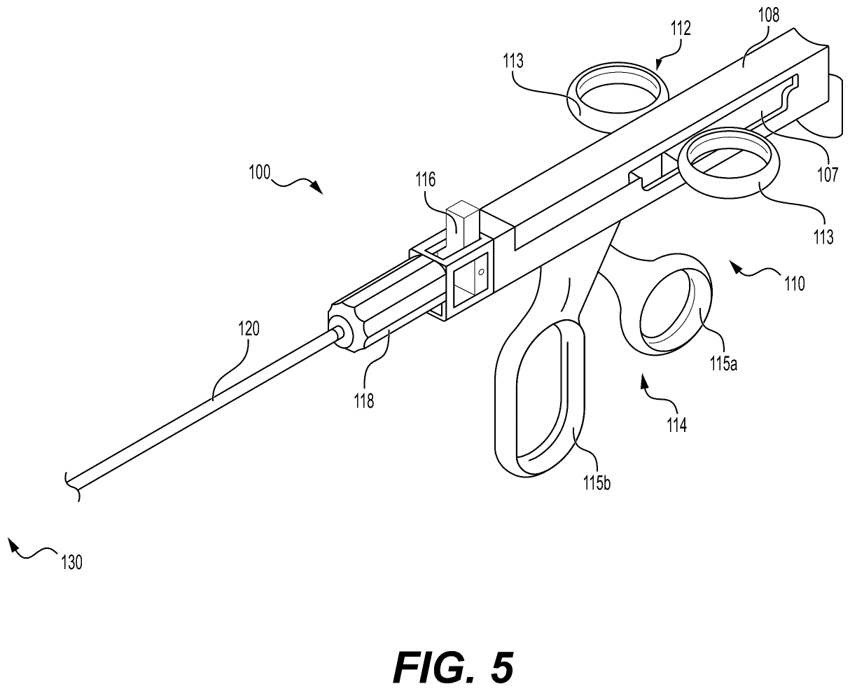

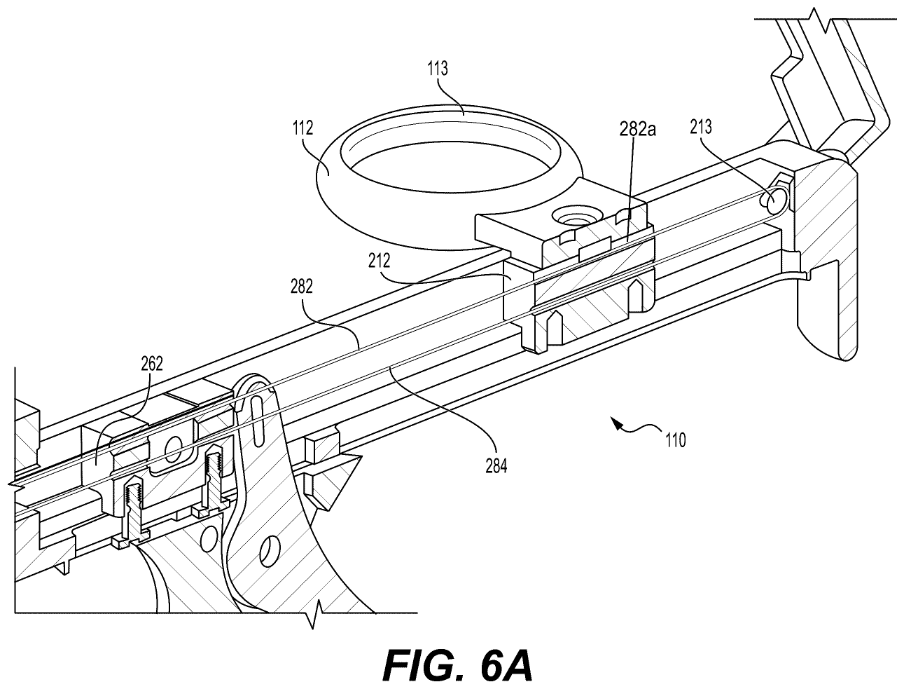

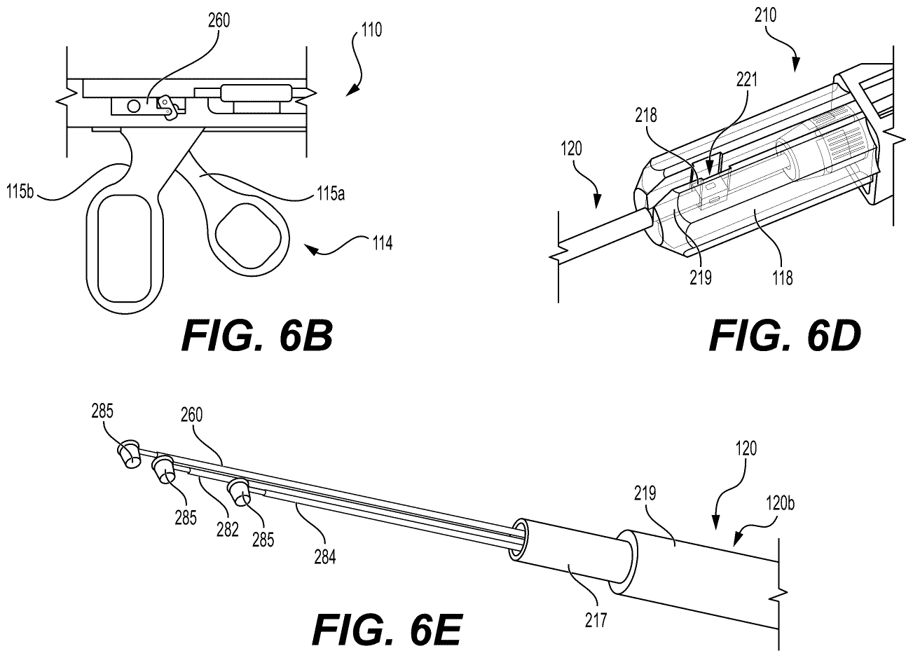

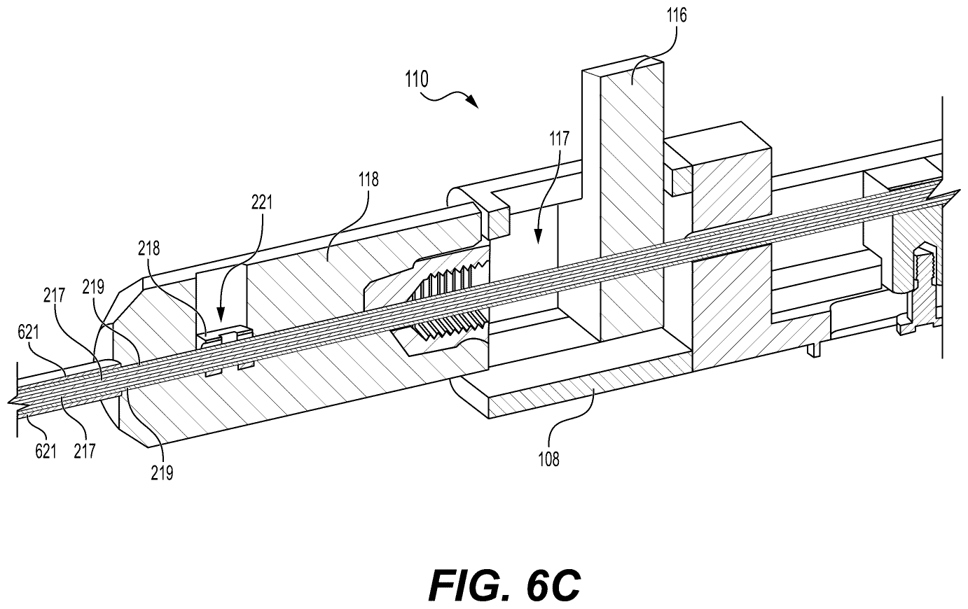

FIGS. 5-6E show features of handle 110 and shaft 120. Handle 110 may be configured to be gripped by a hand or hands of an operator (i.e., may be hand-held). Handle 110 of medical device 100 may include a body 108 and various actuators for controlling aspects of shaft 120 and/or end effector 130. As shown in FIG. 5, handle 110 may include a slider 112, which may be a first actuator that is movable relative to body 108. Handle 110 may also include a trigger 114, which may be a second actuator that is movable relative to body 108. Handle 110 may further include a tab 116, which may be a third actuator that is movable relative to body 108. Handle 110 may additionally include a knob 118, which may be a fourth actuator that is movable relative to body 108. Although ordinal numbers, such as “first,” “second,” “third,” etc. are used to describe actuators of handle 110 herein, it will be appreciated that such terms are merely for reference and that any ordinal number may be applied to any of the actuators. Furthermore, the types of actuators described herein are merely exemplary, and any alternative type of actuator may be used. The figures depicting aspects of handle 110 may omit certain features in order to depict details related to a particular actuator or other aspect of handle 110.

Slider 112 may include one or more finger loops 113 (e.g., two finger loops 113) for receiving fingers of an operator. Slider 112 may be longitudinally (i.e., axially) movable in a direction that is approximately coaxial with or parallel to a central longitudinal axis of handle 110. For example, slider 112 may move longitudinally along a slot 107 of body 108. As shown in FIG. 6A, slider 112 may include a coupler 212, which may be a body that extends into an interior of body 108. Slider 112 may be coupled to one or more sled actuation wires/cables 282, 284 (or other actuation/control members). In some examples, cable 282 may be coupled to cable 182, either directly or via an intermediate cable or other structure, and cable 284 may be coupled to cable 184, either directly or via an intermediate cable or other structure. For example, as shown in FIG. 6E, cables 282 and 284 may terminate in respective nubs (protrusions) 285 at distal ends of cables 282, 284, which may be removably (or fixedly) couplable to respective rings 185 of cables 182, 184. Such couplings may occur proximally, near handle 110 or distally, near end effector 130. In other examples, cable 282 may be continuous with (the same structure as) cable 182, and/or cable 284 may be continuous with (the same structure as) cable 184.

As shown in FIG. 6A, proximal ends of cable 282 and cable 284 may be coupled to one another (e.g., may be a single, unitary structure or may be separate pieces that are fixed to one another) and may extend around a pulley 213. Whereas cables 182, 184 form a distal loop, cables 282, 284 may form a proximal loop, with the proximal loop extending around/passing over pulley 213. As shown in FIG. 6A, for example, pulley 213 may be at a proximal end (e.g., a proximalmost end) of body 108.

Coupler 212 may be fixedly coupled to one of cables 282, 284 (e.g., cable 282 at a coupler 282a), and the other of cables 282, 284 (e.g., cable 284) may movably extend through coupler 212 (e.g., through a lumen of coupler 212). Thus, as slider 112 moves proximally and/or distally along body 108, coupler 212 may move the cable 282, 284 to which it is fixedly attached (e.g., cable 282) proximally and/or distally. Because cables 282 and 284 are coupled to one another and extend over pulley 213, the other of cables 282, 284 (e.g., cable 284) may move in the opposite direction. For example, if slider 112 moves cable 282 proximally (applying tension to cable 282), then cable 284 may move distally (releasing tension from cable 284), through coupler 212. If slider 112 moves cable 282 distally, then cable 284 may move proximally, through coupler 212. Cables 282 and 284 may, in turn, move cables 182 and 184, respectively, as described above. In such a manner, slider 112 may control proximal and distal movement of sled 170. Proximal (backward) movement of slider 112 may move sled 170 proximally (backward), and distal (forward) movement of slider 112 may move sled 170 distally (forward).

As mentioned, handle 110 may further include trigger 114. Trigger 114 may include a fixed ring 115a and a movable ring 115b, as shown in FIGS. 5 and 6B. Movable ring 115b may be coupled to a linkage 262. Linkage 262 may be coupled to a cable 260 (FIG. 6B) or other actuation/control member, which may be continuous with cable 160 (formed of a single, integral piece) or may be a separate piece/member from cable 160 that is coupled to cable 160. A user may insert one or more fingers (e.g., a thumb) through fixed ring 115a and other fingers (e.g., an index finger and/or other fingers) through movable ring 115b. The user may then squeeze movable ring 115b toward fixed ring 115a (e.g., proximally), thereby moving linkage 262 and cable 260 proximally (applying tension to cable 260). Proximal movement of cable 260 may, in turn, move cable 160 proximally, thereby closing/lowering anvil 152 onto cartridge 162. Upon a user removing a force on movable ring 115b, resilient member 159 may cause cable 160 to move distally (as discussed above), which may thereby also move cable 260 and linkage 262 distally and move movable ring 115b away from fixed ring 115a. Alternatively, a user may exert a force on movable ring 115b to move it away from fixed ring 115a (i.e., distally), thereby moving linkage 262, cable 260, and cable 160 distally, and also opening/raising anvil 152.

As mentioned, handle 110 may further include tab 116, which may be used to articulate/bend shaft 120. FIG. 6E depicts a partial tearaway view of proximal portion 120b of shaft 120, to reveal elements of shaft 120. Distal portion 120a of shaft 120 may have similar layers as proximal portion 120b. For example, as shown in FIG. 6E, shaft 120 may include an inner sheath 217 and an outer sheath 219. Sheaths 217 and 219 may be first and second sheaths. Although not shown in FIG. 6E, shaft 120 may also include a cover 621 (shown in FIG. 6C) covering outer sheath 219. As described above, cables 260, 282, 284 may extend through inner sheath 217. Cables 260, 282, 284 may each have nubs 285 for coupling cables 260, 282, 284 to cables 160, 182, 184. For example, nubs 285 may couple to respective rings 185 of cables 160, 182, 184.

Tab 116 may be fixedly coupled to outer sheath 219. Details of tab 116 are shown in FIG. 6C. Tab 116 may be configured to move proximally and distally with respect to body 108 (e.g., along or within a slot 117 in body 108). Outer sheath 219 and inner sheath 217 may be axially (longitudinally) fixed relative to one another at their distal ends (or another portion of sheaths 217, 219) but may be movable with respect to each other at other portions. Movement of tab 116 proximally and distally relative to body 108 may cause shaft 120 to bend, as a result of respective movement between outer sheath 219 and inner sheath 217. In some examples, portions of outer sheath 219 or inner sheath 217 may include slits, openings, or other structures (not shown) to define a bending direction of shaft 120. For example, outer sheath 219 and/or inner sheath 217 may be configured to allow for left and right movement of end effector 130. Tab 116 may have a frictional engagement with body 108, such that tab 116 is self-locking at various positions within slot 117. Thus, a user may select an amount by which to deflect shaft 120. Alternatively, springs or bayonets may be utilized in order to provide control and/or locking of tab 116. Body 108 may include one or more markings or indications (e.g., along slot 117) to correlate a position of tab 116 with a deflection of end effector 130.

Furthermore, as mentioned above, handle 110 may also include knob 118 at a distal end thereof. Details of knob 118 are shown in FIGS. 6C and 6D. Knob 118 may be rotatable about a central longitudinal axis of shaft 120. Outer sheath 219 may include or be coupled to a crimp 218 (or other protruding portion, such as a nut) fixedly attached thereto. Crimp 218 may be received within a cavity 221 of knob 118. Crimp 218 may have a cubic or rectangular prism shape in some examples, as shown in FIGS. 6C and 6D. Cavity 221 may have a complementary shape, such that crimp 218 is keyed to cavity 221. Thus, cavity 221 (and, in turn, knob 118) may be fixed relative to crimp 218. Thus, as knob 118 is turned, outer sheath 219 may rotate along with knob 118. However, inner sheath 217 may be rotatably movable with respect to outer sheath 219, such that inner sheath 217 does not rotate along with outer sheath 219. Although inner sheath 217 and outer sheath 219 may be longitudinally fixed with respect to one another at one or more points, inner sheath 217 and outer sheath 219 may be rotatably movable with respect to one another. Thus, rotation of outer sheath 219 may not affect actuation of cables 260/160, 282/182, and 284/184.

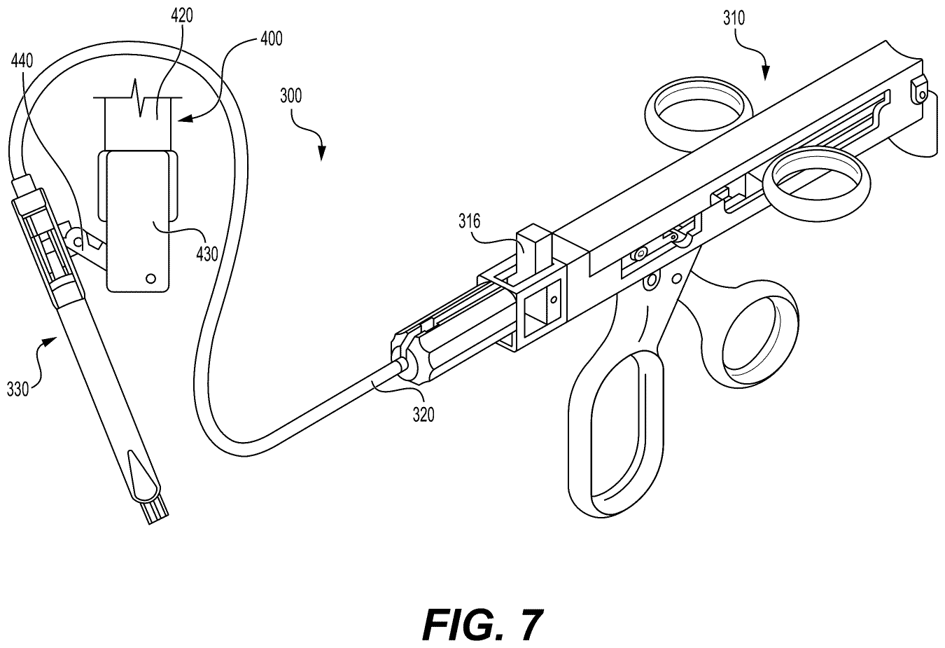

FIG. 7 shows an alternative medical device 300. Unless otherwise specified herein, medical device 300 may include any of the features disclosed above, with respect to medical device 100. Whereas medical device 100 may be configured such that shaft 120 is passed through a working channel of an insertion device (e.g., an endoscope), medical device 300 may be configured such that shaft 120 passes alongside and external to a shaft 420 of an endoscope 400. In other words, medical device 300 may be an over-the-shaft device.

Medical device 300 may include a handle 310, having any of the properties of handle 110. Medical device 300 may also include a shaft 320, having any of the properties of shaft 120. An end effector 330 (having any of the properties of end effector 130) may be disposed at a distal end of shaft 320. Medical device 300 also may include an adapter 430 (attachment device), which may be configured to be coupled to a distal end of endoscope 400. Adapter 430 may be pivotably coupled to a pivot arm 440, which may, in turn, be pivotably coupled to end effector 330. Although end effector 130 of FIG. 1 depicts body 150 including a bracket that may be attached to a pivot arm 440, it will be appreciated that such a bracket may be omitted from end effector 130. Adapter 430, pivot arm 440, and end effector 330 may have any of the features disclosed in U.S. patent application Ser. No. 17/645,529, filed Dec. 22, 2021, and published as U.S. Patent Application Publication No. 2022/0202402.

As described in U.S. patent application Ser. No. 17/645,529, proximal/distal movement of shaft 320 may pivot end effector 330 relative to adapter 430/endoscope 400. In some alternative examples, a tab 316 of handle 310 may be coupled to shaft 320 and may be configured to provide for proximal and distal movement of shaft 320. Whereas shaft 120 included layers configured to move relative to one another, shaft 320 may omit such layers, or such layers may be fixed relative to one another. Tab 316 may be configured to move an entirety of shaft 320 proximally and/or distally in order to pivot end effector 330 relative to adapter 430, thereby articulating end effector 330 left and/or right to change an angle and position of end effector 330 relative to endoscope 400.

In some examples, handle 310 may not include an actuator that correlates to actuator 118. End effector 130 may be rotated about a central longitudinal axis of shaft 410 via rotation of endoscope 400. Alternatively, handle 310 may include an actuator for separately rotating shaft 320.

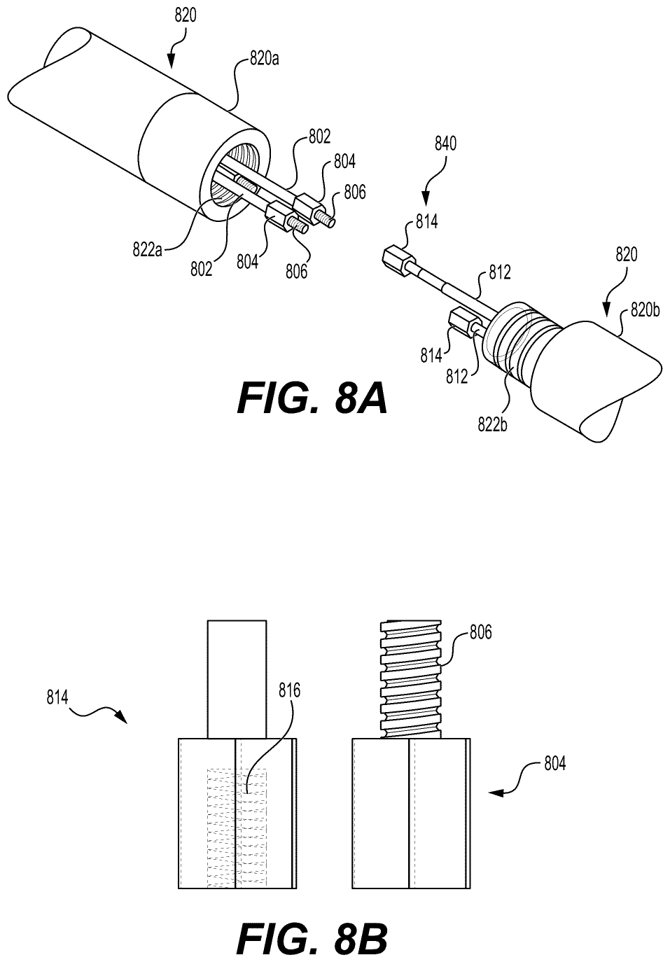

FIGS. 8A and 8B show aspects of a connection mechanism 840 for a first shaft portion 820a (having any of the features of distal portion 120a and/or proximal portion 120b of shaft 120) and a second shaft portion 820b (having any of the features of distal portion 120a and/or proximal portion 120b of shaft 120). It will be appreciated that any of the features of shaft portions 820a, 820b may be reversed and/or combined in any suitable manner.

First shaft portion 820a may have first wire or cable portions 802 extending therethrough. First cable portions 802 may correspond to any of the cables disclosed herein. Although two cable portions 802 are depicted, it will be appreciated that any suitable number of cable portions 802 may be utilized. An end of each cable portion 802 (e.g., a proximal end of cable portions 802 where first shaft portion 820a is a distal portion of shaft 820, or a distal end of cable portions 802 where first shaft portion 820a is a proximal portion of shaft 820) may include a connector 804. Connector 804 may include a threaded post 806 extending therefrom.

Similarly, second shaft portion 820b may have second wire or cable portions 812 extending therethrough. Second cable portions 812 may correspond to any of the cables disclosed herein. Although two cable portions 812 are depicted, it will be appreciated that any suitable number of cable portions 812 may be utilized. An end of each cable portion 812 (e.g., a proximal end of cable portions 812 where second shaft portion 820b is a distal portion of shaft 820, or a distal end of cable portions 812 where second shaft portion 820b is a proximal portion of shaft 820) may include a connector 814. Connector 814 may include a threaded receptacle 816 (FIG. 8B). Threaded receptacles 816 may be configured to removably receive threaded posts 806 in order to releasably connect each of first cable portions 802 to a respective one of second cable portions 812. Cable portions 802, 812 may be color-coded or otherwise include indicators to indicate which of cable portions 802 is to be connected to which of cable portions 812. Additionally or alternatively, directions of rotation (clockwise versus counter-clockwise) may differ for different first cable portions 802/second cable portions 812 to assist with connection of appropriate first cable portions 802/second cable portions 812.

An end of first shaft portion 820a (e.g., a proximal end of first shaft portion 820a, where first shaft portion 820a is a distal shaft segment, or a distal end of first shaft portion 820a, where first shaft portion 820a is a proximal shaft segment) may include a threaded inner surface 822a. An end of second shaft portion 820b (e.g., a proximal end of second shaft portion 820b, where shaft portion 820b is a distal shaft segment, or a distal end of second shaft portion 820b, where second shaft portion 820b is a proximal shaft segment) may include a threaded projection 822b. An outer surface of projection 822b may include threads thereon. Projection 822b may be sized and shaped so as to be received within first shaft portion 820a, so that threads of projection 822b engage with threads of inner surface 822a to releasably secure first shaft portion 820a to second portion 820b. For example, an outer diameter of projection 822b may be approximately the same as an inner diameter of a portion of first shaft portion 820a having threaded inner surface 822a. In other words, first shaft portion 820a may include a female connector, and second shaft portion 820b may include a male connector.

In some examples, connection mechanism 840 may be disposed on a portion of a shaft (e.g., shaft 120 or shaft 320), such that connection mechanism 840 is not inserted into a human body and/or an endoscope. Connection mechanism 840 may be relatively larger than distal portions of shaft 120, 320 to assist with connecting of first shaft portion 820a and second shaft portion 820b. Distal portions of shaft 120, 320 may have an outer diameter of approximately 2.0 mm to approximately 2.6 mm.

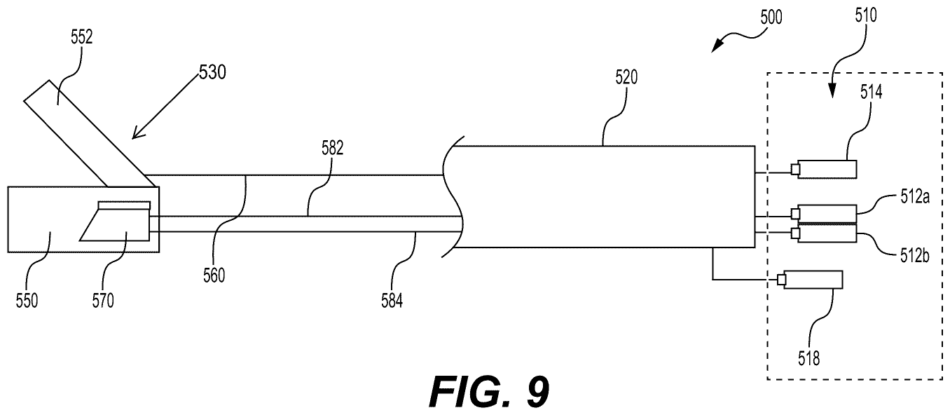

FIG. 9 depicts a schematic diagram of an alternative motorized system 500. Motorized system 500 may include an operation portion 510, one or more shafts 520, and an end effector 530. Unless specified herein, operation portion 510 may have any of the features of handles 110, 310. Similarly, unless specified here, shaft 520 may have any of the properties of shafts 120, 320, 820, and end effector 530 may include any of the features of end effectors 130, 330.

Operation portion 510 may be a single unit (e.g., a handle) or may be comprised of multiple separate elements. In some examples, operation portion 510 may be configured to be gripped by one or more hands of an operator (hand-held). In other examples, operation portion may be a free-standing structure configured to be disposed on a table or other surface, such as an arm or bracket attached to a patient table, or endoscope cart. Operation portion 510 may help to decrease fatigue of an operator because the operator is not required to operate one or more mechanical actuators.

End effector 530 may include a base 550 (having any of the properties of base 150) and an anvil 552 (having any of the properties of anvil 152). A cartridge (e.g., cartridge 162) received in base 550 may include a sled 570, having any of the properties of sled 170, described above. Wires or cables 582, 584 (having any of the properties of cables 182, 184 or the other cables disclosed herein) may be coupled to sled 570. Cables 582, 584 may be used to translate sled 570 proximally and/or distally, as described above for sled 170. A wire or cable 560 may be coupled anvil 552 to open/close anvil 552, as explained above for anvil 152.

Operation portion 510 may include a plurality of motors for controlling various aspects of system 500. For example, a first motor 512a and a second motor 512b may be configured to actuate (apply tension to or release tension from—i.e., detension) cables 582, 584 respectively. Actuation of cables 582, 584 may cause sled 570 to move proximally or distally, depending on which of cables 582, 584 is tensioned/detensioned. For example, tensioning of cable 582 and detensioning of cable 584 may cause sled 570 to move proximally, according to the same principles as described above for sled 170. Detensioning of cable 582 and tensioning of cable 584 may cause sled 570 to move distally.

Operation portion 510 may also include a third motor 514, which may be configured to selectively actuate cable (tension/detension) cable 560. Applying tension to cable 560 may cause anvil 552 to close, as described above for anvil 152 and cable 160. Detensioning/relieving tension on cable 560 may cause anvil 552 to open. In some examples, motors 512a, 512b, and 514 may include linear actuators, which push and/or pull cables 560, 582, 584 proximally or distally in order to tension or detension the cables. Alternatively, motors 512a, 512b, and 514 may be rotational drive systems, which may reel in or release cables 560, 582, 584 as needed. The actuators may be controlled by (e.g., may be elements of) the respective motors.

Operation portion 510 may also include a fourth motor 518 which may be coupled to shaft 520, so as to cause shaft 520 to rotate about a central longitudinal axis of shaft 520, according to any suitable mechanism. For example, fourth motor 518 may be coupled to a cable, which may be coupled to shaft 520. Alternatively, fourth motor 518 may be directly coupled to shaft 520.

The motors described above are merely exemplary, and additional or fewer motors may be utilized. For example, additional motors may be utilized in order to articulate shaft 520, as described above for shaft 120.

In examples, operation portion 510 may be disposable. In other aspects, operation portion 510 may be reusable and may make use of any of the features described herein (e.g., the connection mechanism 800) in order to releasably connect shaft 520 to operation portion 510. Motors of operation portion 510 may be powered by one or more disposable or rechargeable batteries. Alternatively, operation portion 510 may plug into an external power source (e.g., a wall socket or a controller for use with an endoscope).

As opposed to a laparoscopic medical device/system, an endoscopic medical device/system such as medical system 500 may include a flexible shaft 520, which may have a varying, curved (e.g., tortuous) path. Thus, motors 512a, 512b, 514, 518 may compensate for/accommodate elasticity of flexible components (e.g., shaft 520) and changes in length of cables 560, 582, 584 and shaft 520 as they pass along curved paths of a subject's anatomy. In some examples, motors 512a, 512b, 514, 518 may pre-tension when shaft 520 has been inserted into a body lumen and positioned at a working location. Medical system 500 may have sensors at proximal, distal, or both ends of cables 560, 582, 584 to monitor position, force, speed, acceleration, or other characteristics of elements of medical system 500.

A user may operate motors 512a, 512b, 514, 518 via control elements on operation portion 510 itself or via a remote control element (e.g., a video game-style controller or other types of controller), a mobile device, or any other suitable mechanism.

Medical system 500 may also include software that may control an entire fastening procedure. For example, the software may control positioning of end effector 130 (e.g., at a proper angle), pretensioning cables of system 500, closing and locking anvil 152 against body 150/a cartridge (such as cartridge 162). Such software may be stored on a hard drive and executed by a processor, and may monitor motion of aspects of system 500 (e.g. of cables 560, 582, 584, via the sensors described above) and current draw of motors 512a, 512b, 514, 518. Such software may control the various motors 512a, 512b, 514, 518 and/or share information as appropriate to ensure the medical procedure proceeds smoothly and without complications.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device for tissue fastening, the medical device comprising:
an end effector having:
a cartridge;
a sled positioned in the cartridge;
a first control member coupled to the sled; and
a second control member coupled to the sled;
a shaft coupled to the end effector; and
an operation portion coupled to the shaft, wherein the operation portion includes an actuator that is configured to actuate at least the first control member or the second control member in order to move the sled proximally or distally.

2. The medical device of claim 1, wherein the actuator is configured to move the first control member proximally and the second control member distally in order to move the sled proximally.

3. The medical device of claim 2, wherein the actuator is configured to move the first control member distally and the second control member proximally in order to move the sled distally.

4. The medical device of claim 2, wherein the first control member and the second control member are coupled to one another at their distal ends.

5. The medical device of claim 4, wherein the first control member extends along a top surface of a wall of a body of the end effector, and wherein the second control member extends along a bottom surface of the wall of the body.

6. The medical device of claim 5, wherein the body is configured to removably receive the cartridge.

7. The medical device of claim 4, wherein the first control member is fixedly coupled to a bottom surface of the sled.

8. The medical device of claim 1, wherein the sled includes a blade and a slider, and wherein the slider is configured to deliver a fastener to a tissue of a subject.

9. The medical device of claim 1, wherein the actuator is a first actuator, wherein the operation portion includes a second actuator, and wherein the second actuator is configured to articulate the shaft.

10. The medical device of claim 9, wherein the second actuator is fixedly coupled to a first sheath of the shaft, and wherein at least portions of the first sheath are movable with respect to a second sheath of the shaft.

11. The medical device of claim 1, wherein the operation portion includes a motor, and wherein the actuator is controlled by the motor.

12. The medical device of claim 11, wherein the motor is a first motor, wherein the actuator is a first actuator, wherein the operation portion includes a second motor that controls a second actuator, wherein the first actuator is configured to actuate the first control member, and wherein the second actuator is configured to actuate the second control member.

13. The medical device of claim 12, wherein the operation portion includes a third motor that controls a third actuator, wherein the third actuator is configured to actuate a third control member that is coupled to the end effector to close the end effector.

14. The medical device of claim 13, wherein the operation portion includes a fourth motor that is coupled to the shaft and is configured to rotate the shaft, and wherein one or more of the first, the second, or the third motors is configured to accommodate a changing length of at least one of the first control member, the second control member, or the third control member.

15. The medical device of claim 1, wherein the operation portion is releasably coupled to the shaft.

16. A medical device for tissue fastening, the medical device comprising:
an end effector having:
a cartridge;
a sled positioned in the cartridge;
a cable coupled to the sled, wherein the cable has two proximal ends and a looped distal end;
a shaft coupled to the end effector; and
an operation portion coupled to the shaft, wherein the operation portion includes an actuator that is coupled to the proximal ends of the cable in order to actuate the sled proximally and distally.

17. The medical device of claim 16, wherein the cable is a first cable, wherein the actuator is fixedly coupled to a second cable, and wherein the second cable has two distal ends and a looped proximal end.

18. The medical device of claim 17, wherein the distal ends of the second cable are coupled to the proximal ends of the first cable.

19. A medical device for tissue fastening, the medical device comprising:
an end effector having:
an anvil; and
a cartridge that contains a plurality of fasteners;
a flexible shaft coupled to the end effector; and
an operation portion releasably coupled to the flexible shaft.

20. The medical device of claim 19, wherein the operation portion includes at least one motor for controlling the end effector.
